(19) United States
(12) Patent Application Publication
(10) Pub. No.: US 2025/0375289 A1
(43) Pub. Date: Dec. 11, 2025

(54) SKIRT FOR IMPLANTABLE MEDICAL DEVICE

(71) Applicant: Greatbatch Ltd., Clarence, NY (US)

(72) Inventors: Brendan Aspel, CO. Kildare (IE); Dean King, Cavan (IE)

(21) Appl. No.: 19/229,253

(22) Filed: Jun. 5, 2025

Related U.S. Application Data
(60) Provisional application No. 63/657,142, filed on Jun. 7, 2024.

(51) Int. Cl. A61F 2/24; A61F 2/00

(52) U.S. Cl. CPC A61F 2/2409; A61F 2002/0081; A61F 2210/0057; A61F 2210/0076; A61F 2230/0086; A61F 2250/0069

(57) ABSTRACT

In various examples, a skirt for creating a seal between an implantable vascular device and vascular anatomy of a patient includes a seamless tubular structure. The tubular structure is formed from a textile material. A plurality of protrusions extend radially outwardly from the tubular structure. The protrusions are formed within and integral with the tubular structure. Each protrusion including an initial shape, wherein the protrusions are configured to flatten with pressure applied in a radially inward direction with respect to the tubular structure and recoil with pressure at least partially removed to regain the initial shapes of the protrusions, such that the protrusions conform to the vascular anatomy to create the seal between the implantable vascular device and the vascular anatomy.

### CROSS REFERENCE TO RELATED APPLICATION

This application claims the benefit of priority to U.S. Provisional Application Ser. No. 63/657,142, filed on Jun. 7, 2024, entitled “THE USE OF THERMOFORMED TEXTILES FOR THE REDUCTION IMPLANT RELATED LEAKAGE,” which is incorporated by reference herein in its entirety.

### BACKGROUND

Cardiovascular and structural heart devices are typically deployed directly against the native tissue. Due to the anatomically diverse patient population, non-concentric nature of vasculature, and the presence of confounding co-morbidities, an idealized fit of an implantable vascular device relative to a native anatomy of a patient is rarely achieved.

For instance, transcatheter aortic valve implantation (TAVI) systems specifically are deployed over the native leaflets, making the surface underneath the implant less than ideal for a flush fit to be achieved. The objective of a valvular replacement is to replace the native valve as it has become damaged, stenotic, calcified, or the like and can no longer function properly causing it to occlude and/or regurgitate blood during the cardiac cycle.

An artificial valve is implanted to reestablish the valve's ability to control the direction of blood flow. When the leaflets of the new prosthetic valve close and the native cardiac muscle contracts, chamber pressures in the heart increase. If there are gaps between the outside of the valve and the native anatomy, then a backflow of blood around the valve will occur. This is called paravalvular regurgitation or paravalvular leakage (PVL).

Depending on the heart valve that was replaced and the study cohort, up to 17% of patients treated with a TAVI experience PVL, with up to 5% being clinically significant. As a result, PVL has become a primary technical challenge that the market is trying to address in their next generation valvular replacement systems.

The current preferred approach to managing PVL is to use a high-volume cloth and/or rubber inflatable rings around the outside of the implant so when the system is deployed it presses against the native anatomy, reducing, if not eliminating, the presence of gaps or voids around the valve. However, including such textiles and/or thick components significantly increases the bulk volume of the crimped valve assembly, which, in turn, requires designers to use a larger catheter to reduce the delivery forces associated with the larger, bulkier valvular assembly. Failure to use a larger catheter can result in damaging the implant during implantation and/or significantly impacting useability for clinicians due to the high forces required to deliver to the therapeutic area.

### OVERVIEW

This overview is intended to provide an overview of subject matter of the present patent document. It is not intended to provide an exclusive or exhaustive explanation of the invention. The detailed description is included to provide further information about the present patent document.

The present inventors have recognized, among other things, that the present subject matter can be used to provide a skirt configured to seal an implantable medical device within native anatomy of a patient. In some examples, the present invention is advantageous in that it provides a skirt for sealing between an implantable heart valve and the vascular of the patient to lessen, if not eliminate, paravalvular leakage (PVL). The present invention is further advantageous in that it provides, in some examples, a skirt that is collapsible to allow for the implantable medical device to fit within a relatively small-diameter catheter delivery system. In further examples, the present invention lessens, if not eliminates, PVL without the drawbacks associated with causing a significant bulk increase of the final valve assembly, thus reducing, if not eliminating, the risk of PVL without requiring a larger, redesigned catheter delivery system. To better illustrate the devices and methods described herein, a non-limiting list of examples is provided here:

Example 1 can include subject matter that can include a skirt for creating a seal between an implantable vascular device and vascular anatomy of a patient. The skirt includes a seamless tubular structure. The tubular structure is formed from a textile material. A plurality of protrusions extend radially outwardly from the tubular structure. The protrusions are formed within and integral with the tubular structure. Each protrusion includes an initial shape, wherein the protrusions are configured to flatten with pressure applied in a radially inward direction with respect to the tubular structure and recoil with pressure at least partially removed to regain the initial shapes of the protrusions, such that the protrusions conform to the vascular anatomy to create the seal between the implantable vascular device and the vascular anatomy.

In Example 2, the subject matter of Example 1 is optionally configured such that the textile material includes a thermoformed textile material.

In Example 3, the subject matter of Example 1 or 2 is optionally configured such that the plurality of protrusions includes pleats extending along the tubular structure.

In Example 4, the subject matter of any one of Examples 1-3 is optionally configured such that the plurality of protrusions includes rings extending at least partially around a circumference of the tubular structure.

In Example 5, the subject matter of any one of Examples 1-4 is optionally configured such that the plurality of protrusions includes discrete bumps at least partially covering the tubular structure.

In Example 6, the subject matter of Example 5 is optionally configured such that the discrete bumps include frustopyramidal structures.

In Example 7, the subject matter of any one of Examples 1-6 optionally includes a coating disposed on at least a portion of the tubular structure.

In Example 8, the subject matter of Example 7 is optionally configured such that the coating is a spray coating.

In Example 9, the subject matter of Example 7 or 8 is optionally configured such that the tubular structure with the coating applied thereto includes a coated radial force profile which is different than an uncoated radial force profile of the tubular structure without the coating applied thereto.

In Example 10, the subject matter of Example 9 is optionally configured such that the coated radial force profile is greater than the uncoated radial force profile.

Example 11 can include, or can optionally be combined with any one of Examples 1-10 to include subject matter that can include an implantable medical device including an implantable vascular device configured to be implanted within vascular anatomy of a patient. A skirt is attached to the implantable vascular device. The skirt includes a seamless tubular structure. The tubular structure is formed from a textile material. A plurality of protrusions extend radially outwardly from the tubular structure. The protrusions are formed within and integral with the tubular structure, each protrusion including an initial shape. The protrusions are configured to flatten with pressure applied in a radially inward direction with respect to the tubular structure and recoil with pressure at least partially removed to regain the initial shapes of the protrusions, such that the protrusions conform to the vascular anatomy to create a seal between the implantable vascular device and the vascular anatomy.

In Example 12, the subject matter of Example 11 is optionally configured such that the textile material includes a thermoformed textile material.

In Example 13, the subject matter of Example 11 or 12 is optionally configured such that the plurality of protrusions includes pleats extending along the tubular structure.

In Example 14, the subject matter of any one of Examples 11-13 is optionally configured such that the plurality of protrusions includes rings extending at least partially around a circumference of the tubular structure.

In Example 15, the subject matter of any one of Examples 11-14 is optionally configured such that the plurality of protrusions includes discrete bumps at least partially covering the tubular structure.

In Example 16, the subject matter of Example 15 is optionally configured such that the discrete bumps include frustopyramidal structures.

In Example 17, the subject matter of any one of Examples 11-16 optionally includes a coating disposed on at least a portion of the tubular structure, wherein the tubular structure with the coating applied thereto includes a coated radial force profile which is different than an uncoated radial force profile of the tubular structure without the coating applied thereto.

In Example 18, the subject matter of any one of Examples 11-17 is optionally configured such that the implantable vascular device includes a replacement heart valve.

Example 19 can include, or can optionally be combined with any one of Examples 1-18 to include subject matter that can include a skirt for creating a seal between an implantable vascular device and vascular anatomy of a patient. The skirt includes a seamless tubular structure. The tubular structure is formed from a thermoformed textile. A plurality of protrusions extends radially outwardly from the tubular structure. The protrusions are formed within and integral with the tubular structure. The plurality of protrusions includes at least one of pleats extending along the tubular structure, rings extending at least partially around a circumference of the tubular structure, and discrete bumps at least partially covering the tubular structure. Each protrusion includes an initial shape, wherein the protrusions are configured to flatten with pressure applied in a radially inward direction with respect to the tubular structure and recoil with pressure at least partially removed to regain the initial shapes of the protrusions, such that the protrusions conform to the vascular anatomy to create the seal between the implantable vascular device and the vascular anatomy.

In Example 20, the subject matter of Example 19 optionally includes a coating disposed on at least a portion of the tubular structure, wherein the tubular structure with the coating applied thereto includes a coated radial force profile which is different than an uncoated radial force profile of the tubular structure without the coating applied thereto.

### BRIEF DESCRIPTION OF THE DRAWINGS

FIG. 1 is a perspective view of a skirt for an implantable medical device, the skirt being in accordance with at least one example of the invention.

FIG. 2 is a side view of the skirt of FIG. 1.

FIG. 3 is an end view of the skirt of FIG. 1.

FIG. 4 is a cross-sectional view of the skirt of FIG. 1 taken along line 4-4 of FIG. 2.

FIG. 5 is an enlarged view of protrusions of the skirt of FIG. 4.

FIG. 6 is a perspective view of a skirt for an implantable medical device, the skirt being in accordance with at least one example of the invention.

FIG. 7 is a perspective view of a skirt for an implantable medical device, the skirt being in accordance with at least one example of the invention.

FIG. 8 is a perspective view of a skirt for an implantable medical device, the skirt being in accordance with at least one example of the invention.

FIG. 9 is a perspective view of an implantable medical device including a skirt in accordance with at least one example of the invention.

FIG. 10 is a perspective view of the implantable medical device of FIG. 9.

FIG. 11 is an end view of the implantable medical device of FIG. 9.

FIG. 12 is a side view of the implantable medical device of FIG. 9.

FIG. 13 is a cut-away perspective view of the implantable medical device of FIG. 9 disposed within vasculature of a patient.

FIG. 14 is a cut-away side view of the implantable medical device of FIG. 9 disposed within vasculature of a patient.

FIGS. 15A and 15B are perspective views of the making of a skirt for an implantable medical device, the skirt being in accordance with at least one example of the invention.

FIG. 16 is a perspective view of a device for forming protrusions in a textile structure for a skirt for an implantable medical device, the skirt being in accordance with at least one example of the invention.

FIGS. 17A-17D are views of a top plate of the device of FIG. 16.

FIGS. 18A-18D are views of a bottom plate of the device of FIG. 16.

### DETAILED DESCRIPTION

The present invention relates generally to an implantable medical device. More specifically, the present invention relates to a skirt for an implantable medical device, the skirt being configured to seal the implantable medical device within native anatomy of a patient. In some examples, the present invention provides a skirt for sealing between an implantable heart valve and the vasculature of the patient to lessen, if not eliminate, paravalvular leakage (PVL). In some examples, the present invention provides a skirt that is collapsible to fit within a relatively small-diameter catheter delivery system. In further examples, the present invention lessens, if not eliminates, PVL without the drawbacks associated with causing a significant bulk increase of the final valve assembly, thus reducing, if not eliminating, the risk of PVL without requiring a larger, redesigned catheter delivery system.

In some examples, the present inventive subject matter improves the fit of the deployed implant relative to the unpredictable, non-uniform native anatomy of the patient without increasing risk of tissue trauma or perforation. In some examples, this allows the device to function closer to its as-engineered state, improving mechanical performance and overall clinical outcomes without requiring a full redesign and/or revalidation of existing catheter delivery systems.

In some examples, the skirt of the present inventive subject matter does not significantly increase in the crimped assembly bulk volume. Therefore, in some examples, unlike the conventional approaches, the inclusion of the skirt with the implantable device does not require the redesign of the delivery system to accommodate a significant increase in overall valve system size. In some examples, the use of a thin thermoformed textile for the skirt increases a volume of the device post-deployment while maintaining a relatively small pre-deployment size of the implantable medical device to allow for implantation using a relatively small catheter delivery system.

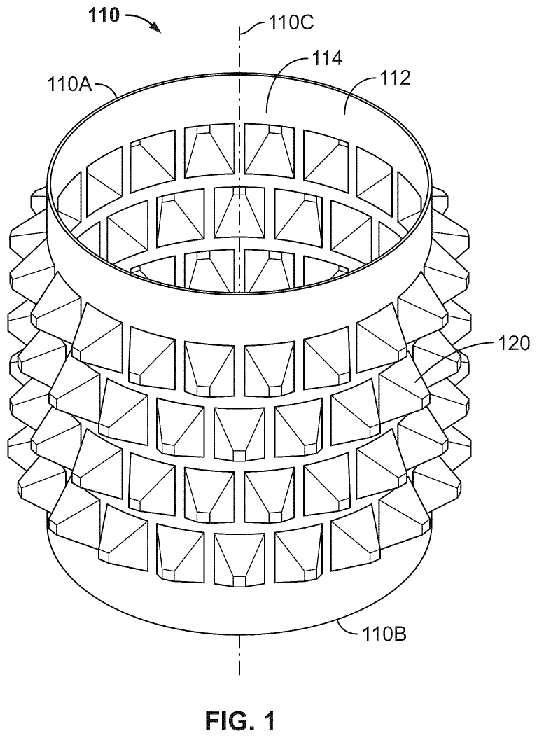

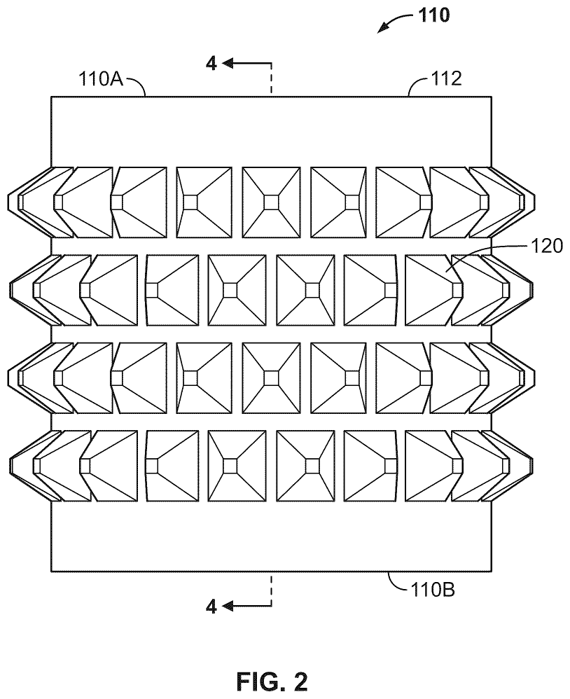

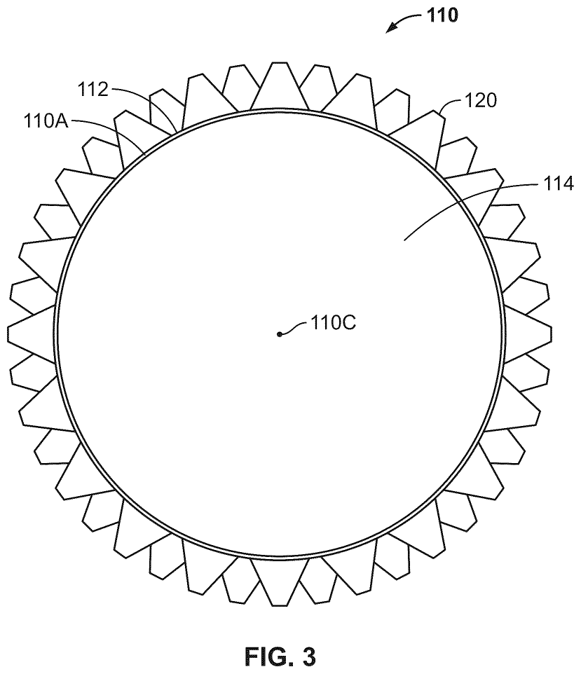

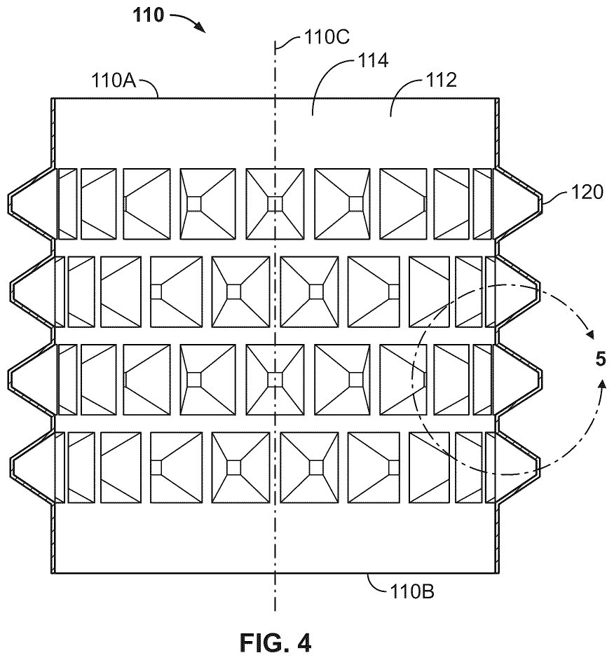

Referring to FIGS. 1-5, in some examples, a skirt 110 is configured to be used with an implantable medical device in order to create a seal between the implantable medical device and an anatomy of a patient. The implantable medical device, in some examples, includes an implantable vascular device. In some examples, the skirt 110 is configured to create a seal between the implantable vascular device and a vascular anatomy of a patient. For instance, in some examples, the skirt 110 is attached to a replacement heart valve device, which, when deployed in position at a site of a heart valve being replaced, the skirt 110 expands to substantially fill any space between the replacement heart valve and a native anatomy of the patient in order to reduce, if not eliminate, PVL. Oftentimes, the native anatomy of the patient is irregular and uneven. In such circumstances, in some examples, the skirt 110 is configured to compress to varying degrees in some areas (for instance, where there is a protuberance of the native anatomy extending into the deployment position of the implantable medical device). In this way, in some examples, the skirt 110 substantially conforms to the native anatomy of the patient to form a seal between the native anatomy of the patient and the implantable medical device in order to reduce, if not eliminate, PVL. In some examples, the present inventive subject matter includes a technology that can be applied to either a flat textile structure or a tubular or other complex geometric textile structure to include features that are capable of being flattened when pressure is applied but, in turn, are capable of recoiling and recovering their initial geometric shape once the applied pressure is either partially or fully removed. In this way, in some examples, the present inventive subject matter can occlude gaps around a structural heart, cardiovascular, or endovascular implant and, in turn, improve the seal that is achievable relative to the geometry of the native anatomical structures.

In some examples, the skirt 110 includes a substantially tubular structure 112. In some examples, the tubular structure 112 is seamless in that it includes a continuous tube of material. In other examples, the tubular structure 112 includes a seam running a length of the tubular structure 112 where two ends of a previously-flat piece of material are joined to form the tubular structure 112. In some examples, the tubular structure 112 is formed from a textile material. The textile material, in some examples, includes various configurations, including, but not limited to, a knitted textile, a woven textile, a non-woven textile, a braided textile, or a combination thereof. In further examples, the textile material can include one or more of one or more polymeric materials, one or more metallic materials, and/or one or more natural materials that may be present homogenously or in combination with other materials. In some examples, the skirt 110 includes a seamless textile tubular structure.

The skirt 110, in some examples, includes a thin, flexible material forming the substantially tubular structure 112, the skirt 110 including a first opening 110A at a first side of the skirt 110 and a second opening 110B at a second side of the skirt 110. The skirt 110, in some examples, includes a longitudinal axis 110C extending along the length of the skirt 110 from the first opening 110A to the second opening 110B. An interior 114, in some examples, is defined within the tubular structure 112, the interior 114 being configured to accommodate an implantable medical device therein, as will be described in more detail herein.

The skirt 110, in some examples, is collapsible or compressible into a smaller form with an inward force or pressure applied to the skirt 110 and then capable of expanding back to an original shape of the skirt 110 when the inward force or pressure is ceased or removed. In some examples, the material forming the substantially tubular structure 112 includes shape memory characteristics to allow for the skirt 110 to be compressed from an initial shape of the tubular structure 112 and then be capable of reverting back to the initial shape thereafter when uncompressed.

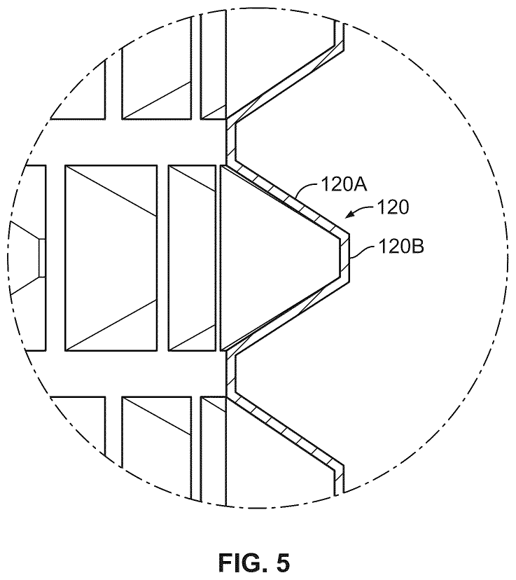

In some examples, the skirt 110 includes a plurality of protrusions 120 extending radially outwardly from the tubular structure 112. That is, in some examples, the protrusions 120 extend outwardly from the tubular structure 112 in a direction that is substantially perpendicular to the longitudinal axis 110C of the skirt 110. In some examples, the protrusions 120 are formed within and integral with the tubular structure 112. In some examples, each protrusion 120 includes an initial shape. In various examples, the protrusions 120 can take various geometric forms including, but not limited to, pleats, rings, and/or pyramidical structures, either in localized regions of the textile material that forms the skirt 110 or across an entire area of the textile material that forms the skirt 110. In some examples, one or more of the protrusions 120 include discrete bumps. In some examples, the skirt 110 includes the discrete bump protrusions 120 at least partially covering the tubular structure 112. In further examples, the skirt 110 includes the discrete bump protrusions 120 at least substantially covering the tubular structure 112. In some examples, at least some of the discrete bump protrusions 120 include substantially pyramidal structures. In some examples, referring particularly to FIG. 5, the substantially pyramidal structure protrusions 120 include pitched sides 120A extending from the tubular structure 112 and a substantially flat end 120B disposed radially outwardly from the tubular structure 112. That is, in some examples, the substantially pyramidal structure protrusions 120 are truncated pyramidal-shaped structures, otherwise known as frustopyramidal structures. In some examples, the substantially flat end 120B is configured to seal against the native anatomy better than protrusions having other shapes because the flat end 120B covers a greater surface area and is capable of conforming better to the native anatomy than protrusions having smaller (for instance, pointed) ends. In still further examples, the substantially pyramidal structure protrusions 120 having the substantially flat end 120B are atraumatic to the native anatomy when placed within the vasculature of the patient. In some examples, the pyramidal structure protrusions 120 include four pitched sides 120A. In other examples, the pyramidal structure protrusions 120 can include three pitched sides or more than four pitched sides, depending upon the desired characteristics of the pyramidal structure protrusions 120. In still other examples, the discrete bump protrusions 120 can include other geometries, such as, for instance, rounded bumps, rectangular prismatic bumps, pointed pyramidal bumps, conical bumps, or frustoconical bumps, to name a few.

In other examples, either instead of or in addition to the discrete bump protrusions 120, the skirt 110 can include protrusions in the form of pleats extending longitudinally along the tubular structure 112, radially around the tubular structure 112, or extending from the tubular structure 112 at an angle therebetween; annular rings extending at least partially around a circumference of the tubular structure 112; a spirally-oriented ring extending along and around the tubular structure 112; or a combination thereof.

In some examples, the plurality of protrusions 120 act to help the skirt 110 seal against the native anatomy of the patient, which, as described above, is often irregular and non-uniformly shaped. In some examples, the protrusions 120 are configured to flatten or push radially inwardly toward the tubular structure 112 with pressure applied in a radially inward direction with respect to the tubular structure 112. In this way, in some examples, any protrusions 120 in the area of irregular anatomy compress to varying degrees depending upon the amount that the irregular anatomy features extend into the space within which the skirt 110 is disposed, thereby closing any gaps between the skirt 110 and the native anatomy and, in turn, substantially sealing the area between the skirt 110 and the native anatomy. In some examples, the protrusions 120 then recoil with pressure at least partially removed to regain the initial shapes of the protrusions 120. In this way, in some examples, the protrusions 120 are configured to conform to the vascular anatomy to create the seal between the implantable vascular device and the vascular anatomy.

In some examples, the tubular structure 112 of the skirt 110 is seamless, such that the skirt 110 is formed from a continuous piece of textile material formed into the tubular structure 112. This configuration is an alternative to other examples in which the tubular structure 112 includes a seam (generally in the axial direction along the length of the tubular structure 112) where ends of a flat piece of textile material are attached together to form the tubular structure 112 of the skirt 110. In some examples, the seamless tubular structure 112 can include additional advantages over the seamed tubular structure 112 in that the seam of the tubular structure 112 can result in an area of different rigidity of the tubular structure 112 than the areas other than the seam, resulting in different compression/recoil characteristics of the tubular structure 112 and/or the protrusions 120 in the area of the seam. In some examples, the seam can result in increased rigidity of the tubular structure 112 and/or the protrusions 120 in the area of the seam due to joining techniques (for instance, application of heat to the two sides of the textile material to thermally join the two sides of the textile material together), which can result in lessened compression around irregular native anatomic structures over which the skirt 110 is placed. Such differing performance at the location of the seam can result in a location of leakage between the skirt 110 and the native anatomy. With the seamless skirt 110 of some examples, there is a more uniform rigidity in all areas of the skirt 110 resulting in a more uniform performance (that is, compression/recoiling) of the skirt 110 in sealing against the native anatomy of the patient.

In some examples, the textile material that forms the skirt 110 includes a thermoformed textile material. Thermoforming of the textile material allows the skirt 110 to attain a desired shape and include shape memory for the skirt 110, such that the skirt 110 can be collapsed to a relatively small size (for instance, to enable deployment using a relatively small-diameter introducer, catheter, or other placement device) and then substantially regain the initial shape of the skirt 110 once deployed at the desired location within the patient.

In some examples, the skirt 110 includes a coating disposed on at least a portion of the tubular structure 112. In some examples, the entire tubular structure 112 includes the coating thereon. In some examples, the coating includes a spray coating. The tubular structure 112 with the coating applied thereto, in some examples, includes a coated radial force profile which is different than an uncoated radial force profile of the tubular structure 112 without the coating applied thereto. In some examples, the coated radial force profile is greater than the uncoated radial force profile. That is, in some examples, the amount of force needed to compress the protrusions 120 and/or the tubular structure 112 of the skirt 110 can be tweaked by the amount and/or type of coating used on the tubular structure 112 and/or protrusions 120. For instance, in some examples, the addition of a first coating on the tubular structure 112 and/or the protrusions 120 can increase the rigidity of the tubular structure 112 and/or the protrusions 120, thereby increasing the force or pressure needed to compress the tubular structure 112 and/or the protrusions 120 from that of uncoated tubular structure 112 and/or the protrusions 120. In turn, in other examples, the addition of a second coating on the tubular structure 112 and/or the protrusions 120 can decrease the rigidity of the tubular structure 112 and/or the protrusions 120, thereby decreasing the force or pressure needed to compress the tubular structure 112 and/or the protrusions 120 from that of uncoated tubular structure 112 and/or the protrusions 120.

In some examples, coating, such as, but not limited to spray coating, allows the recoiling nature of protrusions 120 to be further engineered and tailored to achieve a specific radial force profile when pressure is applied. In some examples, the inclusion of a coating can also help mitigate some risks associated with material creep of the tubular structure 112 and the protrusions 120 of the skirt 110 when a load is applied to the skirt 110 for a sustained period of time.

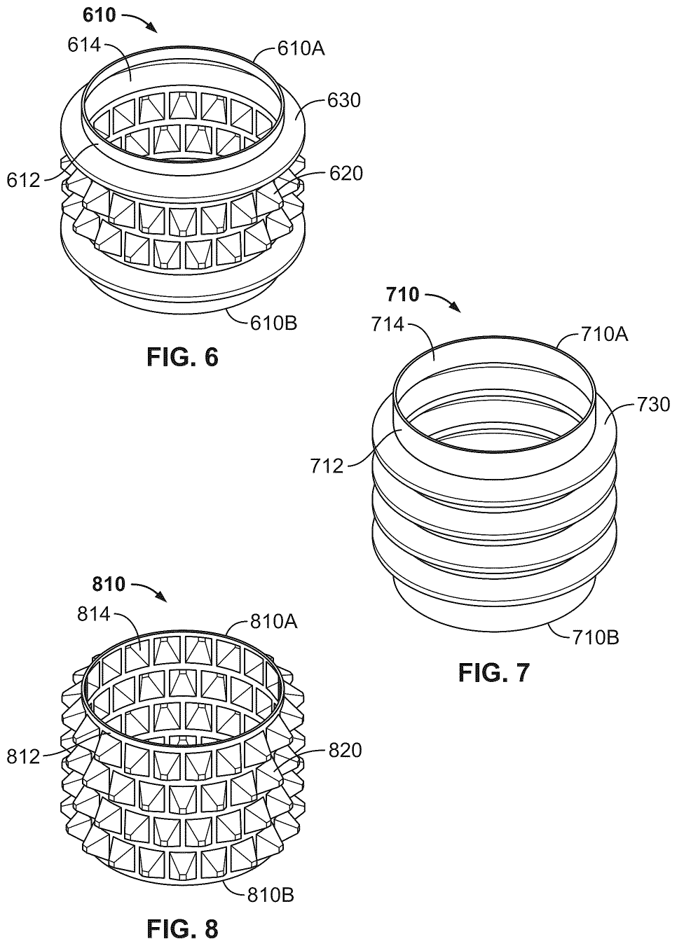

Referring now to FIGS. 6-8, other examples of skirts 610, 710, 810 are shown, which are largely similar to the skirt 110 described and shown herein with differences primarily related to the configurations and shapes of the protrusions 620, 630, 730, 820, as described below. As such, the description of the skirt 110 above is incorporated with respect to each of the descriptions of the skirts 610, 710, 810 below.

Referring specifically to FIG. 6, the skirt 610 includes a substantially tubular structure 612 with a first opening 610A and a second opening 610B and an interior 614 defined within the tubular structure 612. As stated above, the skirt 610, in some examples, is largely similar to the skirt 110 described above; however, the skirt 610 includes differently configured protrusions to those of the skirt 110. That is, in some examples, the skirt 610 includes protrusions 620, 630 extending radially outwardly from the tubular structure 612. In some examples, the protrusions 620, 630 include one or more discrete bump protrusions 620 disposed over at least a portion of the tubular structure 612 and one or more ring protrusions 630 disposed over at least a portion of the tubular structure 612. In the example shown in FIG. 6, the tubular structure 612 includes a plurality of discrete bump protrusions 620 disposed between two ring protrusions 630, one ring protrusion 630 being disposed proximate the first opening 610A and one ring protrusion 630 being disposed proximate the second opening 610B. However, this is not intended to be limiting as the skirt 610, in other examples, can include more or fewer than two ring protrusions 630 and/or discrete bump protrusions 620 disposed proximate one or both of the first and second openings 610A, 610B.

Referring specifically to FIG. 7, the skirt 710 includes a substantially tubular structure 712 with a first opening 710A and a second opening 710B and an interior 714 defined within the tubular structure 712. As stated above, the skirt 710, in some examples, is largely similar to the skirt 110 described above; however, the skirt 710 includes differently configured protrusions to those of the skirt 110. That is, in some examples, the skirt 710 includes protrusions 730 extending radially outwardly from the tubular structure 712. In some examples, the protrusions 730 include one or more ring protrusions 730 disposed over at least a portion of the tubular structure 712. In the example shown in FIG. 7, the tubular structure 712 includes a plurality of ring protrusions 730 disposed between the first opening 710A and the second opening 710B. While four ring protrusions 730 are shown with respect to the skirt 710 of FIG. 7, this is not intended to be limiting as the skirt 710, in other examples, can include more or fewer than four ring protrusions 730.

Referring specifically to FIG. 8, the skirt 810 includes a substantially tubular structure 812 with a first opening 810A and a second opening 810B and an interior 814 defined within the tubular structure 812. As stated above, the skirt 810, in some examples, is largely similar to the skirt 110 described above; however, the skirt 810 includes differently configured protrusions to those of the skirt 110. That is, in some examples, the skirt 810 includes protrusions 820 extending radially outwardly from the tubular structure 812. In some examples, the protrusions 820 include one or more discrete bump protrusions 820 disposed over at least a portion of the tubular structure 812. In the example shown in FIG. 8, the tubular structure 812 includes a plurality of discrete bump protrusions 820 disposed between the first opening 810A and the second opening 810B differing from the discrete bump protrusions 120 of the skirt 110 only in that the discrete bump protrusions 820 are disposed closer to each of the first and second openings 810A, 810B of the skirt 810 than are the discrete bump protrusions 120 to the first and second openings 110A, 110B of the skirt 110. In some examples, various configurations of discrete bump protrusions 820 are contemplated, differing in total number of protrusions 820, numbers of rows of protrusions 820, spacing between adjacent protrusions 820, and proximity to the first and/or second openings 810A, 810B of the skirt 810.

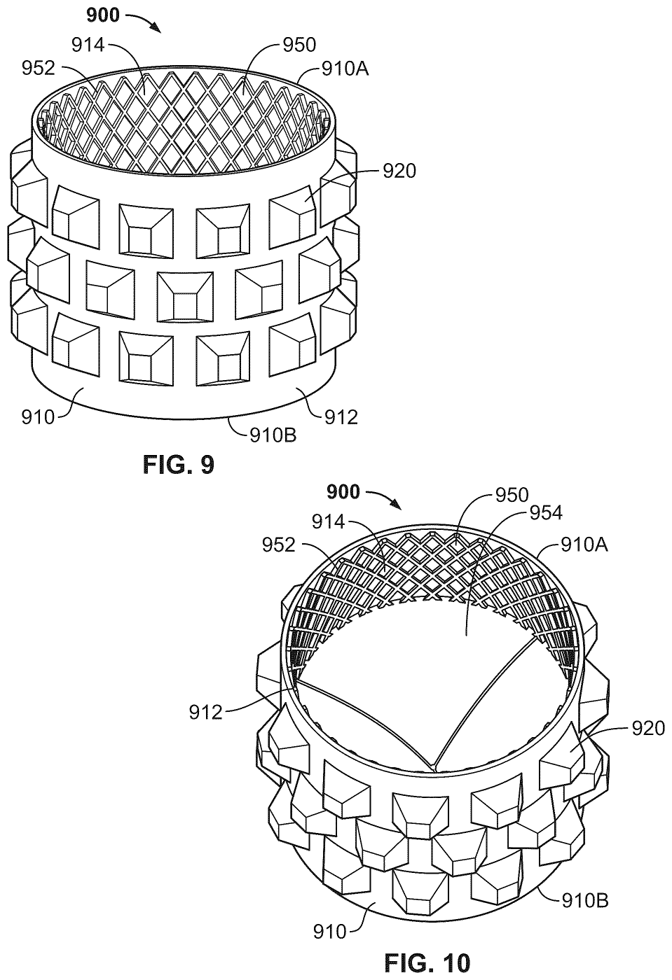

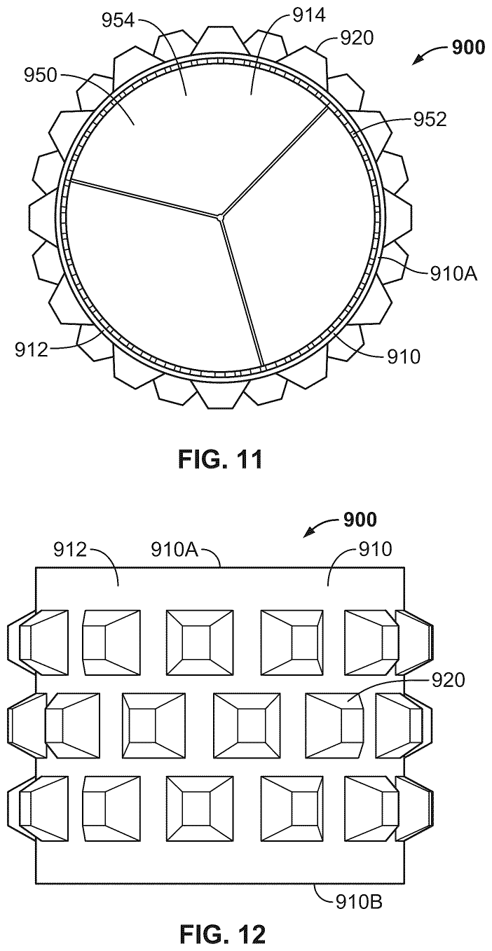

Referring to FIGS. 9-14, in some examples, an implantable medical device 900 includes a skirt 910 attached to an implantable device 950 configured to be implanted within the anatomy of a patient. In some examples, the implantable device 950 includes an implantable vascular device 950 configured to be implanted within the vascular anatomy of the patient. Although the implantable device 950 shown in FIGS. 9-14 includes a valve replacement device 950, this is not intended to be limiting. In various examples, the implantable device 950 can include any cardiovascular device, endovascular device, and/or structural heart device. It is noted that the valve replacement device 950 is very generally shown in FIGS. 9-14 merely for the sake of illustrating the skirt 910 in use with an implantable device 950, such as the valve replacement device 950. As such, the valve replacement device 950, in some examples, includes a frame 952 to which leaflets 954 are attached and are configured to open and close with respect to the beating of a heart of the patient.

In some examples, the skirt 910 is largely similar to the skirts 110, 610, 710, 810 described and shown herein. As such, the descriptions of the skirts 110, 610, 710, 810 above are incorporated with respect to the description of the skirt 910 below.

The skirt 910, in some examples, includes a substantially tubular structure 912 with a first opening 910A and a second opening 910B and an interior 914 defined within the tubular structure 912. In some examples, the tubular structure 912 is formed from a textile material. In some examples, the tubular structure 912 is seamless, such that the tubular structure 912 is formed from a continuous piece of textile material. In other examples, the tubular structure 912 can include a seam substantially longitudinally oriented with respect to the tubular structure 912 where sides of a flat piece of textile material are joined to form the substantially tubular structure 912 of the skirt 910.

In some examples, the skirt 910 includes protrusions 920 extending radially outwardly from the tubular structure 912. In some examples, the protrusions 920 include one or more discrete bump protrusions 920 disposed over at least a portion of the tubular structure 912. In some examples, the tubular structure 912 includes a plurality of discrete bump protrusions 920 disposed between the first opening 910A and the second opening 910B very similar to the discrete bump protrusions 120, 820 of the skirts 110, 810. In some examples, various configurations of discrete bump protrusions 920 are contemplated, differing in total number of protrusions 920, numbers of rows of protrusions 920, spacing between adjacent protrusions 920, and proximity to the first and/or second openings 910A, 910B of the skirt 910. In some examples, at least some of the discrete bump protrusions 920 include substantially pyramidal structures, such as, for instance, frustopyramidal structures. In other examples, the discrete bump protrusions 920 can include geometries other than substantially pyramidal, such as, for instance, rounded bumps, rectangular prismatic bumps, pointed pyramidal bumps, conical bumps, or frustoconical bumps, to name a few.

In still other examples, either instead of or in addition to the discrete bump protrusions 920, the skirt 910 can include protrusions in the form of pleats extending longitudinally along the tubular structure 912, radially around the tubular structure 912, or extending from the tubular structure 912 at an angle therebetween; annular rings extending at least partially around a circumference of the tubular structure 912; a spirally-oriented ring extending along and around the tubular structure 912; or a combination thereof.

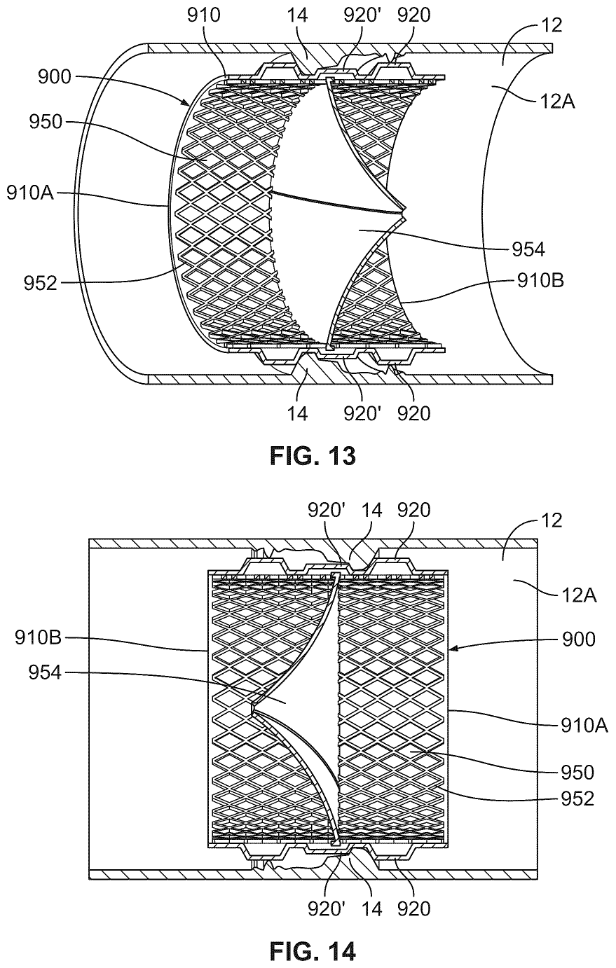

In some examples, the skirt 910 is attached to the implantable device 950. The implantable device 950, in some examples, is disposed within the interior 914 of the skirt 910, with the frame 952 of the implantable device 950 attached to an interior surface of the tubular structure 912 of the skirt 910. In this way, with the skirt 910 disposed around the implantable device 950, the implantable medical device 900 can seal against native anatomy 14 at a location 12A within vasculature 12 of a patient (see FIGS. 13 and 14). In some examples, the native anatomy 14 can include native leaflets of a valve being replaced with the implantable device 950, such as the valve replacement device 950. With deployment of the implantable medical device 900 at the location 12A within the vasculature 12 of the patient, in some examples, the frame 952 of the implantable vascular device 950 expands radially outwardly against the vasculature 12 and the native anatomy 14. With the potentially irregular nature of the native anatomy 14, in some examples, the skirt 910 allows for sealing of the implantable device 900 against the vasculature 12 which otherwise would not have been possible given the substantially cylindrical outer profile of the implantable vascular device 950.

That is, in some examples, the plurality of protrusions 920 act to help the skirt 910 seal against the native anatomy 14 of the patient. In some examples, the protrusions 920 are configured to flatten or push radially inwardly toward the tubular structure 912 with pressure applied in a radially inward direction with respect to the tubular structure 912. In this way, in some examples, any protrusions 920′ in the area of irregular native anatomy 14 compress to varying degrees depending upon the amount that the irregular anatomy features extend into the space within which the skirt 910 is disposed, thereby closing any gaps between the skirt 910 and the native anatomy 14 and, in turn, substantially sealing the area between the skirt 910 and the native anatomy 14. In some examples, the protrusions 920 then recoil with pressure at least partially removed to regain the initial shapes of the protrusions 920. In this way, in some examples, the protrusions 920 are configured to conform to the native anatomy 14 of the vasculature 12 to create the seal between the implantable vascular device 950 and the native anatomy 14.

Various ways of making skirts such as the skirts 110, 610, 710, 810, 910 shown and described herein are contemplated. In some examples, three-dimensional shapes are imparted into a textile material by placing a greige or pre-heatset textile material into tooling that is sized to the final textile geometry. Once the textile material has been positioned appropriately, in some examples, the tooling is closed and heat is applied. In some examples, the textile material can be held at a soak temperature for a period of time before being cooled in a controlled manner. In some examples, this thermal processing of the textile material anneals the constituent structures of the yarns while it is held in this target geometry by the tooling. In this way, in some examples, the constituent yarn structures can be heatset to the geometry of the tooling, such that when the textile material is removed from the tooling, the textile material retains the applied geometry. In some examples, application of pressure in conjunction with heat can impart a slight mechanical stress to the polymer of the textile material that is relieved from the polymeric or equivalent structures during the heating and cooling cycle. This can help apply some complex geometries repeatably to the textile material. Furthermore, in some examples, the inclusion of consumable chemicals can be used during the heat setting process to improve the heat transfer during the heat setting process or further help impart a residual stiffness to the constituent yarns of the textile material.

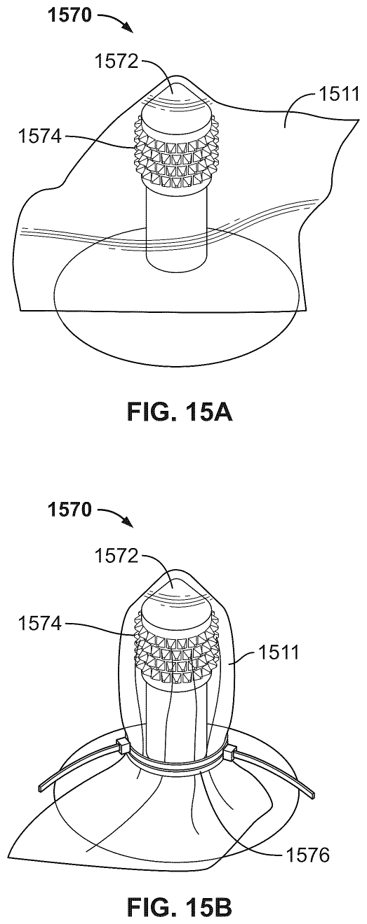

Referring to FIGS. 15A and 15B, in some examples, a forming device 1570 is configured for forming a seamless skirt. In some examples, the forming device 1570 includes a tool 1572 including a patterned portion 1574 thereon. The patterned portion 1574, in some examples, includes the form of the intended protrusions for the skirt. For instance, in the present example, the patterned portion 1574 includes a plurality of pyramidal shapes intended to form a plurality of discrete pyramidal bump protrusions for the skirt (for instance, similar to the protrusions 120, 620, 820, 920 of the skirts 110, 610, 810, 910 described above). In other examples, the patterned portion 1574 can include different shapes (such as, for instance, rounded bumps, rectangular prismatic bumps, pleats, annular rings, a spirally-oriented ring, or the like or a combination thereof) if different protrusions are desired for a particular skirt.

In some examples, a textile material 1511 is placed over the tool 1572 and constrained using, for instance, cable ties 1576 over the tool 1572 so that the cable ties 1576 are offset 180 degrees from each other. The cable ties 1576, in some examples, are then tightened but not fully tightened so as to allow the textile material 1511 to be adjusted. In some examples, the textile material 1511 can be gently pulled down through the cable ties 1576 working around in a circle until a first (top) row of features of the patterned portion 1574 are substantially wrinkle-free. Once substantially wrinkle-free, in some examples, the textile material 1511 over the first row of features of the patterned portion 1574 can be constrained using, for instance, a heavy yarn or other material wrapped around the textile material 1511 over the first row of features of the patterned portion 1574. The process, in some examples, is then repeated for a next (second) row of features of the patterned portion 1574 and a following (third) row of features of the patterned portion 1574 and so on until all rows of features of the patterned portion 1574 are covered with textile material 1511 and constrained. In some examples, the cable ties 1576 and the textile material 1511 can be trimmed and the tool 1572 and the textile material 1511 can be dunked in isopropyl alcohol (IPA) and allowed to soak for a period of time before being removed and drained. The tool 1572 and the textile material 1511, in some examples, can then be loaded into an oven and heated for a period of time while standing upright. Once heating is complete, in some examples, the tool 1572 and the textile material 1511 can be removed from the oven, allowed to cool, and unwrapped leaving one cable tie holding the cloth to the tool to ensure a clean laser cut. In some examples, the top and bottom of the textile material 1511 can be laser cut and the remaining seamless skirt (for instance, similar to one or more of the skirts 110, 610, 710, 810, 910) can be removed from the tool 1572.

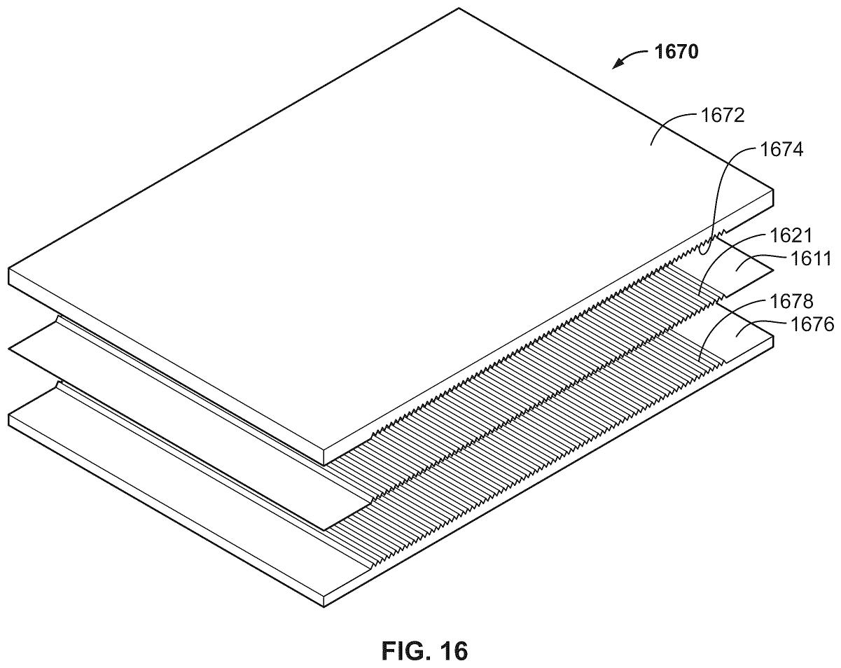

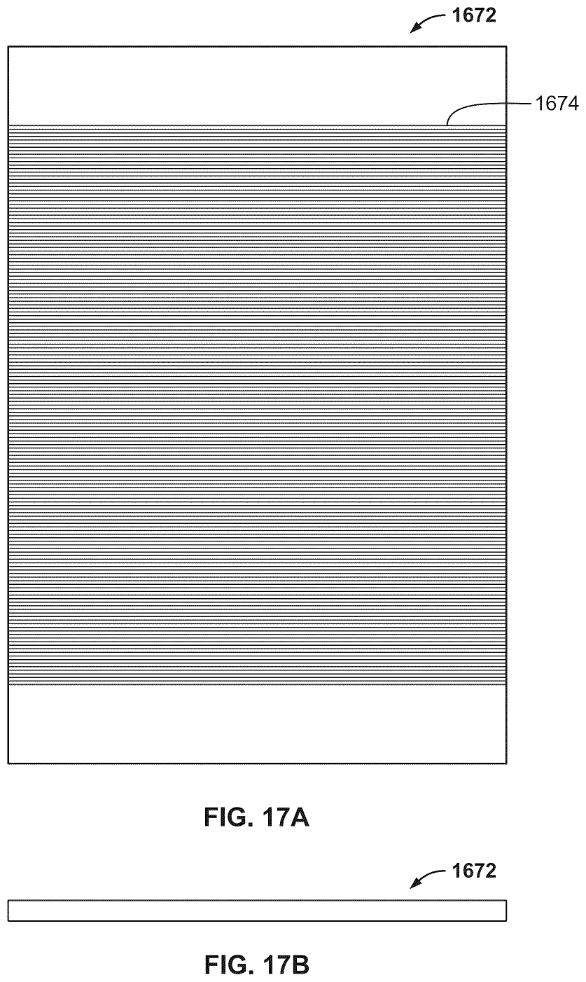

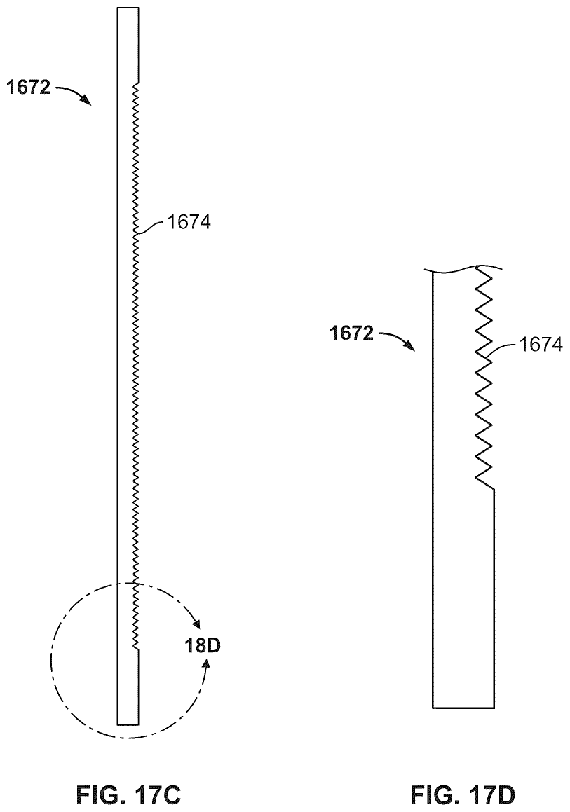

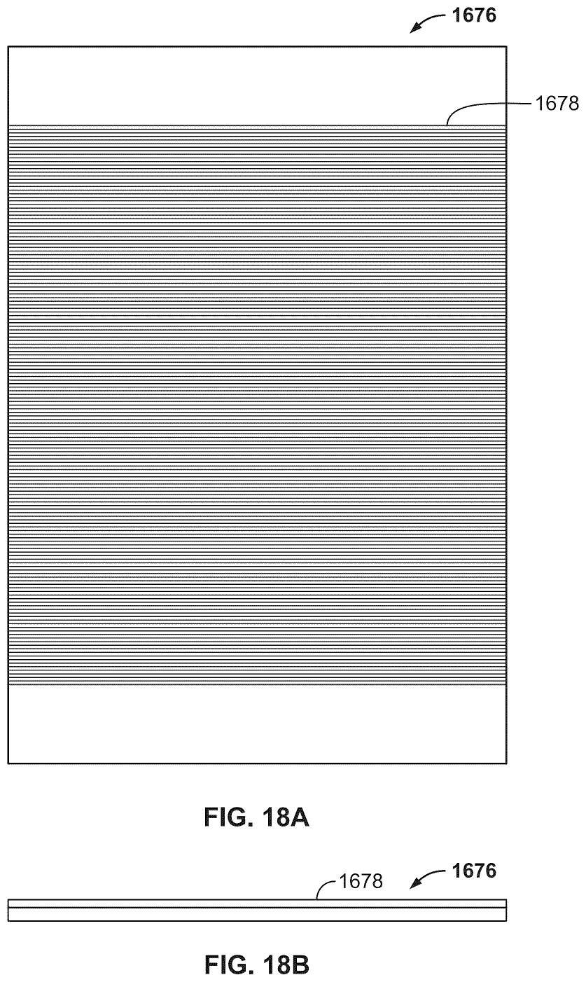

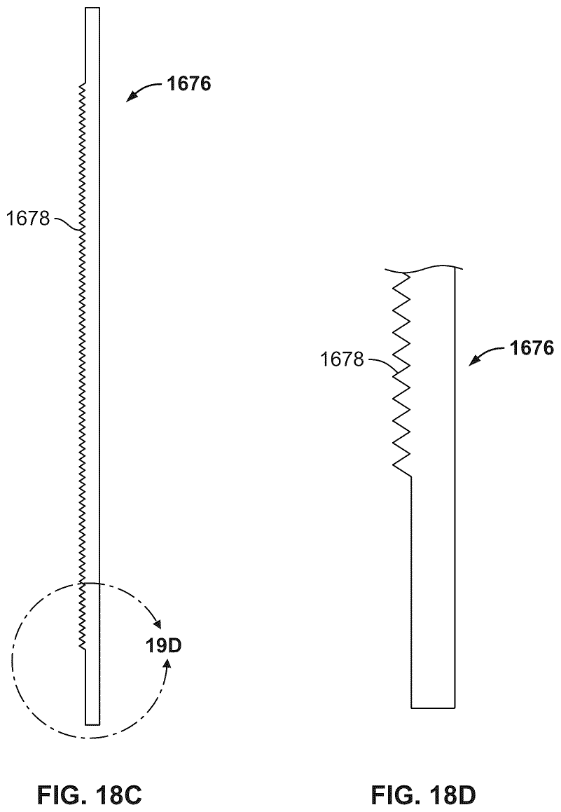

Referring to FIGS. 16-18D, in some examples, a forming device 1670 is configured for forming a seamed skirt. In some examples, the forming device 1670 includes a first plate 1672 including a first patterned portion 1674 thereon and a second plate 1676 including a second patterned portion 1678. In some examples, the first and second patterned portions 1674, 1678 include complementary patterns that mate when the first and second plates 1672, 1676 are placed together. The first and second patterned portions 1674, 1678, in some examples, include the form of the intended protrusions for the skirt. For instance, in the present example, the first and second patterned portions 1674, 1678 include a plurality of sawtooth ridge shapes intended to form a plurality of pleats for the skirt. In other examples, the first and second patterned portions 1674, 1678 can include different shapes (such as, for instance, pyramidal bumps, rounded bumps, rectangular prismatic bumps, annular rings, a spirally-oriented ring, or the like or a combination thereof) if different protrusions are desired for a particular skirt.

In some examples, the forming device 1670 is configured to heatset a textile material 1611 in a pleated state. The textile material 1611 with pleats 1621, in some examples, can then be formed into a pleated skirt and used to reduce PVL without significantly increasing device bulk and, in turn, forces experienced during catheter delivery. In some examples, the textile material 1611 is placed between the first and second plates 1672, 1676 and the first and second plates 1672, 1676 are placed together so that the first and second patterned portions 1674, 1678 interlock together with the textile material 1611 sandwiched in between. The forming device 1670 and the textile material 1611, in some examples, can then be loaded into an oven and heated for a period of time. Once heating is complete, in some examples, the forming device 1670 and the textile material 1611 can be removed from the oven and allowed to cool. Once cooled, in some examples, the textile material 1611 and can be removed from the forming device 1670, leaving the textile material 1611 with pleats 1621 (or one or more other patterns) therein. In some examples, a piece of the textile material 1611 with pleats 1621 can then be cut out from the textile material 1611, rolled into a substantially cylindrical shape, and sides of the piece of the textile material 1611 joined (thermally, adhesively, or otherwise) to form a seamed skirt (for instance, similar to one or more of the skirts 110, 610, 710, 810, 910).

The present inventors have recognized various advantages of the subject matter described herein. For instance, the present inventors have recognized, among other things, that the present subject matter can be used to provide a skirt configured to seal an implantable medical device within native anatomy of a patient. In some examples, the present invention is advantageous in that it provides a skirt for sealing between an implantable heart valve and the vascular of the patient to lessen, if not eliminate, paravalvular leakage (PVL). The present invention is further advantageous in that it provides, in some examples, a skirt that is collapsible to allow for the implantable medical device to fit within a relatively small-diameter catheter delivery system. In further examples, the present invention lessens, if not eliminates, PVL without the drawbacks associated with causing a significant bulk increase of the final valve assembly, thus reducing, if not eliminating, the risk of PVL without requiring a larger, redesigned catheter delivery system. While various advantages of the example systems are listed herein, this list is not considered to be complete, as further advantages may become apparent from the description and figures presented herein.

Although the subject matter of the present patent application has been described with reference to various examples, workers skilled in the art will recognize that changes can be made in form and detail without departing from the scope of the subject matter recited in the below claims.

The above Detailed Description includes references to the accompanying drawings, which form a part of the Detailed Description. The drawings show, by way of illustration, specific examples in which the present apparatuses and methods can be practiced. These embodiments are also referred to herein as “examples.”

The above Detailed Description is intended to be illustrative, and not restrictive. For example, the above-described examples (or one or more elements thereof) can be used in combination with each other. Other embodiments can be used, such as by one of ordinary skill in the art upon reviewing the above description. Also, various features or elements can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

In this document, the terms “a” or “an” are used to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “about” and “approximately” or similar are used to refer to an amount that is nearly, almost, or in the vicinity of being equal to a stated amount.

In the appended claims, the terms “including” and “in which” are used as the plain-English equivalents of the respective terms “comprising” and “wherein.” Also, in the following claims, the terms “including” and “comprising” are open-ended, that is, an apparatus or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

1. A skirt for creating a seal between an implantable vascular device and vascular anatomy of a patient, the skirt comprising:
a seamless tubular structure, the tubular structure being formed from a textile material; and
a plurality of protrusions extending radially outwardly from the tubular structure, the protrusions formed within and integral with the tubular structure, each protrusion including an initial shape, wherein the protrusions are configured to:
flatten with pressure applied in a radially inward direction with respect to the tubular structure; and
recoil with pressure at least partially removed to regain the initial shapes of the protrusions, such that the protrusions conform to the vascular anatomy to create the seal between the implantable vascular device and the vascular anatomy.

2. The skirt of claim 1, wherein the textile material includes a thermoformed textile material.

3. The skirt of claim 1, wherein the plurality of protrusions includes pleats extending along the tubular structure.

4. The skirt of claim 1, wherein the plurality of protrusions includes rings extending at least partially around a circumference of the tubular structure.

5. The skirt of claim 1, wherein the plurality of protrusions includes discrete bumps at least partially covering the tubular structure.

6. The skirt of claim 5, wherein the discrete bumps include frustopyramidal structures.

7. The skirt of claim 1, comprising a coating disposed on at least a portion of the tubular structure.

8. The skirt of claim 7, wherein the coating is a spray coating.

9. The skirt of claim 7, wherein the tubular structure with the coating applied thereto includes a coated radial force profile which is different than an uncoated radial force profile of the tubular structure without the coating applied thereto.

10. The skirt of claim 9, wherein the coated radial force profile is greater than the uncoated radial force profile.

11. An implantable medical device comprising:
an implantable vascular device configured to be implanted within vascular anatomy of a patient; and
a skirt attached to the implantable vascular device, the skirt including:
a seamless tubular structure, the tubular structure being formed from a textile material; and
a plurality of protrusions extending radially outwardly from the tubular structure, the protrusions formed within and integral with the tubular structure, each protrusion including an initial shape, wherein the protrusions are configured to:
flatten with pressure applied in a radially inward direction with respect to the tubular structure; and
recoil with pressure at least partially removed to regain the initial shapes of the protrusions, such that the protrusions conform to the vascular anatomy to create a seal between the implantable vascular device and the vascular anatomy.

12. The implantable medical device of claim 11, wherein the textile material includes a thermoformed textile material.

13. The implantable medical device of claim 11, wherein the plurality of protrusions includes pleats extending along the tubular structure.

14. The implantable medical device of claim 11, wherein the plurality of protrusions includes rings extending at least partially around a circumference of the tubular structure.

15. The implantable medical device of claim 11, wherein the plurality of protrusions includes discrete bumps at least partially covering the tubular structure.

16. The implantable medical device of claim 15, wherein the discrete bumps include frustopyramidal structures.

17. The implantable medical device of claim 11, comprising a coating disposed on at least a portion of the tubular structure, wherein the tubular structure with the coating applied thereto includes a coated radial force profile which is different than an uncoated radial force profile of the tubular structure without the coating applied thereto.

18. The implantable medical device of claim 11, wherein the implantable vascular device includes a replacement heart valve.

19. A skirt for creating a seal between an implantable vascular device and vascular anatomy of a patient, the skirt comprising:
a seamless tubular structure, the tubular structure being formed from a thermoformed textile; and
a plurality of protrusions extending radially outwardly from the tubular structure, the protrusions formed within and integral with the tubular structure, the plurality of protrusions including at least one of pleats extending along the tubular structure, rings extending at least partially around a circumference of the tubular structure, and discrete bumps at least partially covering the tubular structure, each protrusion including an initial shape, wherein the protrusions are configured to:
flatten with pressure applied in a radially inward direction with respect to the tubular structure; and
recoil with pressure at least partially removed to regain the initial shapes of the protrusions, such that the protrusions conform to the vascular anatomy to create the seal between the implantable vascular device and the vascular anatomy.

20. The skirt of claim 19, comprising a coating disposed on at least a portion of the tubular structure, wherein the tubular structure with the coating applied thereto includes a coated radial force profile which is different than an uncoated radial force profile of the tubular structure without the coating applied thereto.
